(19) United States
(12) Patent Application Publication
(10) Pub. No.: US 2025/0177584 A1
(43) Pub. Date: Jun. 5, 2025

(54) POUCH SYSTEM FOR TREATMENT OF A BIOLOGICAL FLUID BY ELECTROMAGNETIC IRRADIATION

(71) Applicant: Maco Pharma, Mouvaux (FR)

(72) Inventors: Cecile BIRCK, Mouvaux (FR); Charly JOURJON, Mouvaux (FR)

(21) Appl. No.: 18/843,714

(22) PCT Filed: Mar. 3, 2023
(86) PCT No.: PCT/EP2023/055504

§ 371 Date: Sep. 4, 2024

(30) Foreign Application Priority Data
Mar. 4, 2022 (FR) FR2201902

(51) Int. Cl. A61L 2/00

(52) U.S. Cl. CPC A61L 2/0047; A61L 2202/181

(57) ABSTRACT

Bag system (1) for treatment of a biological fluid by electromagnetic irradiation, said bag system comprising at least one element consisting of an irradiation bag (2) intended to contain the biological fluid to be irradiated, said irradiation bag (2) being made of a material that is permeable to said electromagnetic radiation, said bag system (1) further comprising at least one protective film (26a, 26b) covering said irradiation bag (2), said protective film (26a, 26b) being made of a gas-barrier polymer material having a permeability to oxygen of less than or equal to 100 cm³/m²/24 h at a temperature of 23° C. and a relative humidity of 50%.

The invention concerns a bag system for treatment of a biological fluid by electromagnetic irradiation, as well as an assembly comprising said bag system and a package.

It applies to the medical and biomedical field, in particular to the field of blood transfusion, and even more specifically to the field of blood treatment.

In transfusion, blood is first collected from a donor and then separated by centrifugation into different blood products such as packed red blood cells, plasma and packed platelets. To improve the quality and safety of these different blood products, they may undergo various treatments, such as leukocyte removal and/or pathogen reduction.

Document WO 2007/076834 describes a process for reducing pathogens such as bacteria, viruses and/or leukocytes from platelet concentrates. In this process, a bag containing a platelet concentrate is irradiated with UV radiation under agitation. This process has the advantage over other pathogen inactivation processes, such as the commercial Intercept (Cerus) and Mirasol (TerumoBCT) processes, of not using a photosensitive agent such as amotosalen or riboflavin.

In these light-based processes, and particularly in the absence of a photosensitive agent, it is important to ensure that the blood product to be treated receives the necessary and sufficient dose to reduce the rate of pathogens to an acceptable level, without damaging the biological properties of the blood product.

Document WO 2008/034476 describes a bag system suitable for implementing the UV radiation pathogen reduction process described in document WO 2007/076834. The bag system essentially comprises an irradiation bag made of ethylene vinyl acetate (EVA) copolymer, connected to a platelet concentrate storage bag made of plasticized polyvinyl chloride (PVC). The bag system also includes tubing made of plasticized PVC.

In this document, it is stated that the absorption of UV light by EVA is influenced by the degree of polymerization and crosslinking of the EVA.

Other intrinsic factors influence the UV transmittance of an EVA sheet, such as the relative proportion of ethylene and vinyl acetate units, film thickness or surface finish. Extrinsic factors affecting UV transmittance include heat, humidity, the presence of oxygen and exposure to UV rays.

After much research, the applicant has identified another, previously undescribed, factor affecting the UV transmittance of an EVA sheet.

The applicant has demonstrated that the UV transmittance of an EVA bag deteriorates through the migration of certain plasticizers from the PVC of the other components of the bag system, as well as through the migration of certain substances from the package in which the bag system is enclosed.

Indeed, bag systems such as the one described in document WO 2008/034476 include components such as connectors and tubing made of PVC plasticized with di-2-ethylhexyl phthalate (DEHP).

DEHP is also known to be an endocrine disruptor, with possible effects on the reproductive, hormonal and immune systems. It is also potentially carcinogenic. It is therefore recommended to eliminate DEHP from medical devices such as bag systems for blood processing.

DEHP-free bag systems for blood processing are known. For example, document JP2003-171288A describes a bag system comprising a filter for removing leukocytes from whole blood and a plurality of bags for containing blood components, connected to said filter. At least one of the bags contains a citrate plasticizer, while the other bags are made of PVC plasticized with a citrate ester or trimellitate ester.

Document EP 2 731 425 B1 describes a phthalate plasticizer-free bag for storing red blood cell products. The bag is made from a polymer material plasticized with 1,2-cyclohexanedicarboxylic acid diisononyl ester (DINCH) and contains a special additive solution.

The PVC bag systems of these two documents are not suitable for electromagnetic irradiation of blood components, and the choice of plasticizer is motivated by the reduction of hemolysis of red blood cells in DEHP-free bags. Rather than removing DEHP from medical devices, document FR 2965812 proposes to suppress migration of DEHP or other plasticizing compounds from a plasticized PVC object such as tubing, by coating the object with a layer of metal oxide such as titanium dioxide.

Also known from document EP 1 972 354 is a bag system for the separation and reduction of pathogens from a biological fluid using a photosensitive agent activatable by ultraviolet radiation, in which the bag or bags containing the photosensitive agent are arranged in a removable opaque overwrap. This overwrap prevents early activation of the photosensitive agent by ambient light.

Finally, document US 2019/0230919 describes kits for reducing the oxygen level in blood or blood components in order to improve their preservation. These kits essentially comprise a closed, oxygen-impermeable outer container; a flexible inner bag designed to receive blood or blood components; and an oxygen absorber placed in said outer container. The inner bag is made of an oxygen-permeable material such as polyvinylidene fluoride, silicone or polyurethane. Document WO 2021/231650 describes similar kits in which the inner bag is made in particular of polyvinyl chloride plasticized with di-2-ethylhexyl phthalate (DEHP), 1,2-cyclohexanedicarboxylic acid diisononyl ester (DINCH) or butyryl trihexyl citrate (BTHC). Due to the closed outer container enclosing the inner bag, these kits are not suitable for the treatment of blood or blood components by electromagnetic irradiation.

In order to preserve the transmittance of an irradiation bag of a bag system, the invention proposes, according to a first aspect, a bag system for the treatment of a biological fluid by electromagnetic irradiation, said bag system comprising at least one element consisting of an irradiation bag intended to contain the biological fluid to be irradiated, said irradiation bag being made of a material that is permeable to said electromagnetic radiation, said system comprising one or more other elements selected from the group consisting of at least one storage bag and a tubing, at least one of said other elements being made of a polymer material formulated with a plasticizer and at least one protective film forming a barrier means to the plasticizer and covering said irradiation bag, said protective film being made of a gas-barrier polymer material having a permeability to oxygen of less than or equal to 100 cm³/m²/24 h at a temperature of 23° C. and a relative humidity of 50%.

According to a second aspect, the invention relates to an assembly for the treatment of a biological fluid by electromagnetic irradiation, comprising a package and a bag system according to the first aspect of the invention, said bag system being sterilely confined in said package.

According to a third aspect, the invention concerns the use of a film made from a gas-barrier polymer material having a permeability to oxygen of less than or equal to 100 cm³/m²/24 h at a temperature of 23° C. and a relative humidity of 50% as a barrier medium to polyvinyl chloride releasable plasticizers.

Further features and benefits will be described in the following description.

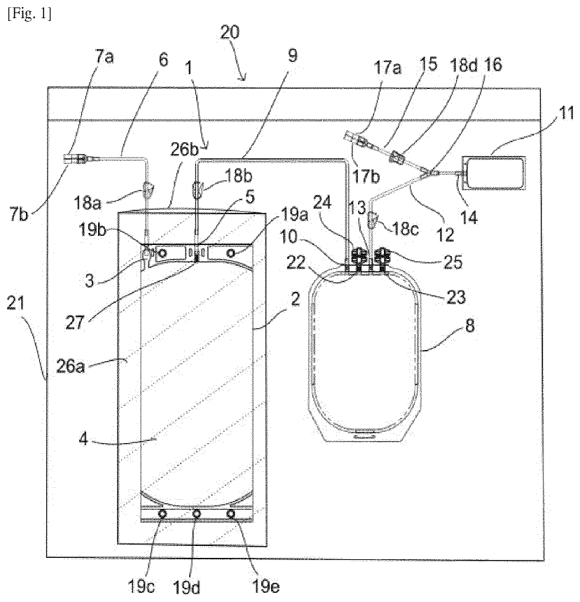

FIG. 1 shows a schematic view of an assembly according to one embodiment of the invention, comprising a sterile package in which a bag system for UV irradiation of a blood product is arranged.

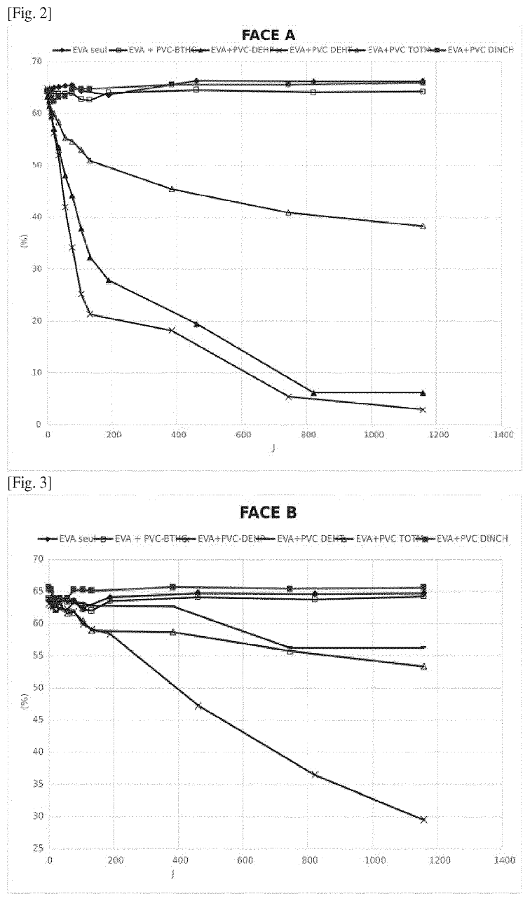

FIG. 2 shows the UV transmittance of an EVA sheet between 0 and 1200 days, below which another EVA sheet is arranged and above which one or two sheets of PVC plasticized with DEHP, DEHT, TOTM or DINCH are superimposed (side A).

FIG. 3 shows the UV transmittance rate of an EVA sheet between 0 and 1200 days, over which another EVA sheet and one or two sheets of PVC plasticized with DEHP, DEHT, TOTM or DINCH are superimposed (side B).

The invention concerns a bag system and an assembly for treatment of a biological fluid by electromagnetic irradiation.

The biological fluid is, in particular, whole blood or a blood product obtained by filtration and/or centrifugation of whole blood, such as plasma, platelet-rich plasma, red cell concentrate or platelet concentrate.

To reduce pathogens such as viruses, bacteria and/or leukocytes in blood or a blood product, it is known to apply electromagnetic radiation to the blood or blood product, in the presence or absence of a photosensitive agent.

By electromagnetic radiation, we mean non-ionizing radiation with wavelengths ranging from 10 nm to 10 μm, i.e. visible, ultraviolet (UV) and/or infrared radiation. In particular, the electromagnetic radiation is visible radiation with wavelengths ranging from 340 nm to 800 nm and/or UV radiation with wavelengths ranging from 200 to 340 nm. More particularly, the radiation is UV-C radiation with wavelengths ranging from 200 to 280 nm. Even more particularly, the UV-C radiation has a wavelength of the order of 254 nm.

According to [FIG. 1], the assembly 20 for treatment by magnetic irradiation comprises a package 21 and a bag system 1 sterilely confined in said package 21.

The package 21 is suitable for sterilizing the bag system 1 in which it is enclosed.

In one embodiment, the package is suitable for sterilization by gases, such as ethylene oxide.

In one embodiment, the package is made from one or more materials formulated without phthalate or terephthalate. For example, the package 21 is formed by assembling a porous non-woven sheet and a transparent film sheet. The porous sheet allows sterilizing gas to pass inside the package, while preventing the passage of microbes into said package. The porous sheet is made, for example, of cellulose nonwoven such as paper or synthetic nonwoven such as high-density polyethylene fiber (Tyvek®). The transparent film sheet is made, for example, of a transparent polymer such as high-density polyethylene, low-density polyethylene, polypropylene and/or polyester. The porous non-woven sheet and the transparent film sheet are welded together, for example.

According to [FIG. 1], the bag system for electromagnetic irradiation of a biological fluid comprises at least one element consisting of an irradiation bag 2 intended to contain the biological fluid to be irradiated. The irradiation bag is made of a material that is permeable to said electromagnetic radiation.

In one embodiment, electromagnetic radiation is UV radiation, in particular UV-C radiation, and the irradiation bag is made of a material that is permeable to UV radiation, in particular UV-C radiation.

The biological fluid to be treated is in particular blood or a blood product such as a platelet concentrate.

For example, the irradiation bag 2 is made of EVA. Alternatively, the irradiation bag 2 can be made of a polymer material permeable to UV radiation, such as polymethylpentene, poly-chloro-trifluoroethylene, perfluoroalkoxy, fluorinated ethylene propylene, ethylene tetrafluoroethylene, ethylene chlorotrifluoroethylene or polyvinylidene fluoride. The material of the irradiation bag is phthalate- and terephthalate-free.

Phthalate or terephthalate refers to a phthalic or terephthalic acid derivative, such as a phthalic or terephthalic acid ester.

A phthalate- and terephthalate-free material is one in which no phthalate or terephthalate as plasticizer has been added to the material composition.

The phthalate- or terephthalate-free formulated material may contain residual traces of phthalate or terephthalate, i.e. a content of less than 1000 ppm, due for example to contamination during the manufacturing process of the material or the bag system, or during its packaging.

In particular, the irradiation bag 2 is formed from a sheath or by assembling two sheets permeable to UV radiation, the sheath or each sheet having a UV-C transmittance greater than 60%, particularly greater than 65%. Even more particularly, the UV-C transmittance of the sheath or of each of the sheets is less than 90%, particularly less than 85%.

UV transmittance is the percentage ratio of the intensity of transmitted radiation to incident intensity. Transmittance is determined spectrophotometrically.

In a particular embodiment, the irradiation bag 2 is free of photosensitive agents. In other words, the irradiation bag 2 does not contain a photosensitive agent such as riboflavin, a psoralen derivative or a phenothiazine derivative.

According to [FIG. 1], the irradiation bag 2 comprises a first access hole 3 in fluid communication with the inner volume 4 of the irradiation bag 2. The irradiation bag 2 further comprises a second access hole 5 in fluid communication with the inner volume 4 of the irradiation bag 2.

The irradiation bag 2 is in fluid communication with a first manifold 6 via the first access port 3. The first manifold 6 is provided at its end with two vents 7a, 7b, each comprising an air-permeable filter membrane forming a sterile barrier. These vents enable the bag system 1 to be sterilized by gas.

The irradiation bag 2 further comprises openings 19a, 19b, 19c, 19d, 19e made in one or more peripheral edges of the irradiation bag 2 for suspending and/or holding said irradiation bag 2.

The bag system 1 also includes a storage bag 8 for collecting and storing the treated blood component. This storage bag 8 is in fluid communication with the irradiation bag 2 via the second tubing 9 that is connected at one end to a second access hole 5 of the irradiation bag 2 and at the other end to an access hole 10 of the storage bag 8. The storage bag 8 also comprises two outlet holes 22,23, each closed by a finned break-off plug 24,25.

A removable plug 27 is arranged in the second access hole 5 of the irradiation bag 2, closing fluid communication between the inner volume 4 of the irradiation bag 2 and the second tubing 9 leading to the storage bag 8. The removable plug 27 can slide out of the second access hole 5, into the inner volume 4 of the container 2, so as to allow fluid communication between the irradiation bag 2 and the storage bag 8.

The bag system 1 further comprises a sampling bag 11 in fluid communication with the storage bag 8 via a third tubing 12 connected at one end thereof to a second access hole 13 of the storage bag 8 and at the other end thereof to an access hole 14 of the sampling bag 11.

A fourth tubing 15 is connected at one end thereof to the third tubing 12 via a three-way connector 16. The other end of this fourth tubing 15 is provided with two vents 17a, 17b, each comprising a filtering membrane that allows air to pass through and forms a sterile barrier.

Each of the tubing 6,9,12,15 is provided with a clamp 18a, 18b, 18c, 18d which allows selective control of the opening and closing of the liquid flow in each of these tubing.

The bag system 1 advantageously comprises at least one protective film 26a covering said irradiation bag. The protective film is a very thin sheet of plastic. It is made of a polymer material formulated without phthalate or terephthalate. The protective film 26a is superimposed on the irradiation bag, by completely covering its surface. The protective film 26a, for example, is positioned between the irradiation bag 2 and the porous sheet, notably made of lacquered paper, of the package 21.

The function of the protective film is to protect the irradiation bag 2 from substances that can adversely affect the transmittance, in particular the UV transmittance, and even more particularly the UV-C transmittance, of said irradiation bag. The irradiation bag 2 is uncovered from this protective film when it is treated with electromagnetic irradiation.

This protective film thus forms a barrier to releasable substances from other elements of the bag system, which adversely affect the transmittance of the irradiation bag. A releasable substance is a substance that migrates from an element of the bag system or its package. Examples of releasable substances are PVC plasticizers, such as phthalates or terephthalates, cyclohexanedicarboxylic acid esters or citrate esters. In other words, the protective film forms a barrier to the plasticizer of a polymer material such as PVC.

Indeed, in its package 21, the bag system 1 is folded in particular so that the storage bag 8 is positioned over the irradiation bag 2. In this configuration, without a protective film, the releasable plasticizers in the bag system migrate towards the irradiation bag 2. The presence of these releasable substances on the irradiation bag deteriorates its optical properties, in particular its UV transmittance, and more specifically its UV-C transmittance.

This protective film also protects the irradiation bag from substances found in the environment that can adversely affect its transmittance. Indeed, phthalates, and DEHP in particular, are present everywhere in the environment and are potential sources of contamination for the irradiation bag.

According to one embodiment, a protective film made of a gas-barrier polymer material having a permeability to oxygen of less than or equal to 100 cm³/m²/24 h at a temperature of 23° C. and a relative humidity of 50% is used as a barrier means to releasable plasticizers from polyvinyl chloride, such as DEHP or DEHT. In particular, the material of the protective film is also a water vapor barrier polymer material with a permeability to water vapor of less than or equal to 10 g/m²/24 h at a temperature of 23° C. and a relative humidity of 50%.

Thus, the protective film 26a is made of a gas-barrier polymer material with a permeability to oxygen of less than or equal to 100 cm³/m²/24 h, more particularly less than or equal to 90, at a temperature of 23° C. and a relative humidity of 50%. The permeability to oxygen is measured in accordance with ASTM D 3985 standard.

In particular, the polymer material of the protective film 26a is a water vapor barrier polymer material with a permeability to water vapor of less than or equal to 10 g/m²/24 h, more particularly less than 5 g/m²/24 h at a temperature of 23° C. and a relative humidity of 50%. The permeability to water vapor is measured in accordance with ASTM F 1249 standard.

In particular, the protective film comprises at least one layer made of polyester or polypropylene. For example, the protective film comprises a composite multilayer sheet comprising one layer made of polyester and one layer made of polypropylene. Alternatively, the protective film comprises a composite multilayer sheet comprising two layers made of polyester or two layers made of polypropylene.

Advantageously, the protective film is transparent, allowing the user to see the irradiation bag 2.

The thickness of the protective film is between 40 and 70 μm, in particular between 45 and 65 μm.

In one embodiment, the bag system comprises two protective films 26a, 26b arranged on either side of the irradiation bag 2. The irradiation bag 2 is thus sandwiched between the two protective films 26a, 26b until it is treated with electromagnetic irradiation. Alternatively, the protective film can be folded on itself so as to form a sheet wherein the irradiation bag 2 is interposed.

In another embodiment, the two protective films 26a, 26b together form a sleeve with at least one open end. The open end of the sleeve enables the user to easily slide the sleeve off the irradiation bag 2 just prior to treatment of the biological fluid contained in the irradiation bag. The irradiation bag 2 is arranged in this sleeve so as to envelop it. The sleeve surrounds the irradiation bag 2 to protect it from the migration of plasticizers or other releasable substances affecting the transmittance of the irradiation bag 2.

For example, the sleeve is made by superimposing the two protective films 26a, 26b, and welding together two opposite sides and a transverse side.

In this embodiment, the irradiation bag 2 is protected from releasable substances of the bag system 1 and of the package 21.

To ensure that the optical properties of the irradiation bag 2 of the bag system 1 are preserved, in addition to the irradiation bag 2, the other element(s) of the bag system are made from one or more materials formulated without phthalate or terephthalate.

According to one embodiment, each of said other elements is made of a material or materials selected from the group consisting of a material other than plasticized polyvinyl chloride and a polymer material formulated with at least one plasticizer other than a phthalate such as di-2-ethylhexyl phthalate or a terephthalate such as di-2-ethylhexyl terephthalate.

Indeed, it has been identified by the applicant that phthalate and terephthalate esters such as di-2-ethylhexyl phthalate (DEHP) or di-2-ethylhexyl terephthalate (DEHT) which migrate towards the irradiation bag 2 lower the UV transmittance, more particularly the UV-C transmittance, of the irradiation bag 2.

Thus, each of the other elements of the bag system 1 is advantageously made of a material or materials other than polyvinyl chloride plasticized with di-2-ethylhexyl phthalate or di-2-ethylhexyl terephthalate.

Advantageously and for the same reasons, the package in which the bag system is contained is made of one or more materials formulated without phthalates or terephthalates, in particular without di-2-ethylhexyl phthalate (DEHP) or di-2-ethylhexyl terephthalate (DEHT).

In a particular embodiment, each of the other elements of the bag system is made either of polyvinyl chloride plasticized with a plasticizer selected from the group consisting of a cyclohexanedicarboxylic acid ester, a citrate ester, a trimellitate ester or a mixture thereof, or of a material other than plasticized polyvinyl chloride.

More particularly, each of the other elements of the bag system is made either of polyvinyl chloride plasticized with a plasticizer selected from the group consisting of 1,2-cyclohexanedicarboxylic acid diisononyl, butyryl trihexyl citrate, tri-2-ethylhexyl trimellitate or a mixture thereof, or of a material other than plasticized polyvinyl chloride.

Other elements of the bag system include one or more elements selected from the group consisting of a storage bag 8, a sampling bag 11, tubing 6, 9, 12, 15, a connector 16, a clamp 18a, 18b, 18c, 18d, a vent 7a, 7b, 17a, 17b and combinations thereof.

The rigid elements of the bag system, such as clamps and vents, are made of a material other than plasticized polyvinyl chloride, such as polycarbonate, polyester, polyethylene and/or polypropylene.

For example, the clamps 18a, 18b, 18c, 18d are made of polycarbonate. The vents 7a, 7b, 17a, 17b are made of polypropylene.

The flexible elements of the bag system, other than the irradiation bag 2, are made of polyvinyl chloride plasticized with at least one plasticizer selected from the group consisting of a cyclohexanedicarboxylic acid ester, a citrate ester, a trimellitate ester and a mixture thereof.

The storage bag 8 is made of a gas-permeable material to enable platelet concentrate to be stored for at least 3 to 7 days. For example, the storage bag 8 is made of PVC plasticized with tris(ethylhexyl) trimellitate (or tri-octyl trimellitate, TOTM) or butyryl trihexyl citrate (BTHC).

For example, the sampling bag 11 is made of PVC plasticized with 1,2-cyclohexanedicarboxylic acid diisononyl ester (DINCH).

For example, the tubing 6, 9, 12, 15 and the connector 16 are made of DINCH-plasticized PVC.

The components of the irradiation bag 2, the storage bag 8 and the sampling bag 11, such as access holes 3,5,10,13,14, outlet holes 22,23 and plugs 24,25,27, are also advantageously made of one or more materials formulated without phthalate or terephthalate.

In connection with embodiment of the assembly 20 shown in [FIG. 1], a method is now described for implementing the bag system 1 and its package 21.

The package 21 in which the bag system 1 is enclosed is opened before the bag system 1 is removed.

A source bag (not shown) containing a biological fluid, e.g. a platelet concentrate from apheresis or a mixture of platelet concentrates obtained from buffy coats, is sterilely connected to the bag system 1 via the first tubing 6, using a sterile connection device of the TSCD-II type (Terumo).

The biological fluid from the source bag is then transferred by gravity through the first access hole 3 into the inner volume 4 of the irradiation bag 2.

After transfer, the air contained in the inner volume 4 of the irradiation bag 2 is expelled from the irradiation bag 2 through the first access hole 3 by exerting pressure on the irradiation bag. Once the air has been expelled, the first access hole 3 is closed by welding.

These air purging and welding operations are advantageously carried out using the welding device described in document WO2022/029040.

The first tubing 6 is welded and cut to separate the source bag, initially containing the biological fluid, from the bag system 1.

The irradiation bag 2 is then removed from the sleeve formed by the two sheets 26a, 26b. Alternatively, this sleeve is released from the irradiation bag 2 at any other time prior to the irradiation operation.

The irradiation bag 2 is irradiated under agitation with UV light so as to inactivate any pathogens present in the biological fluid.

The removable plug 27 closing the second access hole 5 is pushed into the irradiation bag 2, then the irradiated biological fluid is transferred to the storage bag 8 for storage for up to 5 to 7 days.

After transfer, the second tubing 9 is welded and cut using a hand-held welder so as to separate the container 2 from the rest of the bag system 1.

In one embodiment, a sample of the biological fluid is sent to the sampling bag 11 for analysis. It is separated from the storage bag 8 by welding and cutting the third tubing 12, using a hand-held welder.

### EXAMPLES

### Example 1: Impact of Package and Various Plasticized PVC Components of the Bag System on the UV Transmittance of an EVA Sheath

Six test configurations were carried out and analyzed:
EVA sheath (control);
EVA sheath packaged in a pouch made of two transparent composite films of polyester (PET) and polyethylene (PE);
EVA sheath packaged in a pouch made from two sheets of lacquered paper;
BTHC-plasticized PVC storage bag arranged on top of the EVA sheath, and DEHP-plasticized PVC sampling bag straddling the storage bag and the EVA sheath, the whole being packaged in a pouch made from a transparent composite film of PET and PE and of a sheet of lacquered paper;
EVA sheath protected by a composite polymer sleeve of PET and polypropylene (PP), a TOTM-plasticized PVC storage bag arranged on the protected EVA sheath, and a DEHP-plasticized PVC sampling bag straddling the storage bag and the protected EVA sheath, the whole being packaged in a pouch consisting of a composite transparent film of PET and PE and a sheet of lacquered paper;
BTHC-plasticized PVC storage bag arranged over the EVA sheath, and DEHP-plasticized PVC sampling bag straddling the storage bag and the EVA sheath, the whole being packaged in a pouch consisting of a transparent composite film of bi-oriented PET and polypropylene (PP) and of a sheet of paper.

The EVA sheath has substantially the same dimensions as an irradiation bag.

The sleeve, made from a composite polymer of PET and PP, has a thickness of approximately 55 μm, a permeability to oxygen of less than 91.0 cm³/m²/24 h h at 23° C. and relative humidity of 50%, and a permeability to water vapour of less than 5.0 cm³/m²/24 h at 23° C. and relative humidity of 50%.

Transmittance measurements are carried out using a spectrophotometer (Perkin Elmer Lamdbda650).

TABLE 1

UV-C transmittance (%)

| | Average (%) | Average (%) | Average (%) |
|---|---|---|---|
| | T 0 | T 1 month | T 3 months |
| EVA sheath | 64.61 | 65.09 | 63.76 |
| EVA sheath | 64.61 | 54.90 | 52.70 |
| PET/PE pouch | | | |
| EVA sheath | 64.61 | 59.98 | 56.95 |
| lacquered paper | | | |
| pouch | | | |
| EVA sheath | 64.61 | 61.08 | 57.17 |
| PVC/DEHP bag | | | |
| PVC/BTHC bag | | | |
| PET/PP and | | | |
| lacquered paper | | | |
| pouch | | | |
| EVA sheath | 64.61 | 65.17 | 64.02 |
| PET/PP sleeve | | | |
| PVC/DEHP bag | | | |
| PVC/BTHC bag | | | |
| PET/PP and | | | |
| lacquered paper | | | |
| pouch | | | |
| EVA sheath | 64.61 | 63.75 | 63.22 |
| PVC/DEHP bag | | | |
| PVC/BTHC bag | | | |
| bioriented | | | |
| PET/PP and | | | |
| paper pouch | | | |

These results show the negative impact of a particular packaging pouch, both the transparent film and the lacquered paper sheet, on the UV transmittance of the irradiation bag. This impact is reinforced by the presence of the other elements of the plasticized PVC bag system.

The presence of the protective sleeve ensures that the EVA irradiation bag is not contaminated by PVC plasticizers or by other releasable substances from the package, thus maintaining UV transmittance.

### Example 2: Impact of Plasticizer Migration from a PVC Sheet on the UV Transmittance of an EVA Sheet

Six test configurations were carried out by superimposing rectangular samples of around 60 cm² of sheets of different materials:
two EVA sheet samples;
two EVA sheet samples and two PVC sheet samples plasticized with DEHP;
two EVA sheet samples and two PVC sheet samples plasticized with BTHC;
two EVA sheet samples and one PVC sheet sample plasticized with DEHT;
two EVA sheet samples and one PVC sheet sample plasticized with TOTM;
two EVA sheet samples and one PVC sheet sample plasticized with DINCH.

The EVA sheet in direct contact with the plasticized PVC sheet sample is identified as “side A”, while the EVA sheet underneath side A is identified as “side B”.

UV transmittance of EVA sheets was determined at various times between 0 and 3 years. The results are shown in FIGS. 2 and 3.

### 2.1 Impact of DEHP

A sharp drop was observed for the UV transmittance of the EVA sheet in direct contact with a sample of DEHP-plasticized PVC sheet (side A). By the 14^{th} day of contact, the transmittance of the EVA sheet was below 60%. After two years, the UV transmittance of the EVA sheet was less than 10%.

On side B, the transmittance loss falls below 60% after 100 days.

The EVA sheet slows but does not prevent DEHP migration.

### 2.2 Impact of BTHC and DINCH

Under the conditions of the study, contact of the EVA sheet (sides A and B) with a sample of PVC sheet plasticized with BTHC or DINCH has no influence on the transmittance of the EVA sheet over the 3-year period.

### 2.3 Impact of DEHT

Direct contact of the DEHT-plasticized PVC sheet sample with the EVA sheet (side A) results in an even greater loss of UV transmittance for the EVA sheet than with the DEHP-plasticized PVC sheet.

The impact of DEHT-plasticized PVC sheet is less marked on side B, due to the presence of side A, which slows DEHT migration.

### 2.4 Impact of TOTM

A decrease was observed for UV transmittance of the EVA sheet (side A) in direct contact with a sample of PVC sheet plasticized with TOTM, which is however known to migrate little in a PVC matrix, but to absorb UV radiation, particularly UVC. However, this result should be treated with caution, as it represents the average of three tests, two of which showed UV transmittance of the side A at the end of the study to be 55-57%, and one of which showed transmittance of less than 2%. The results on the side B are equally disparate.

These studies show that DEHT, like DEHP, should be avoided in bag systems designed to treat biological fluids with UV irradiation.

### Example 3: Impact of PVC Plasticizer Migration in Tubing on the UV Transmittance of an EVA Sheet

Three test configurations were carried out using a rectangular sample of EVA sheath measuring 15 cm×18 cm, either on its own or superimposed with 60 cm of tubing of different materials, the whole packaged in a pouch made of a transparent composite film of bi-oriented PET and polypropylene and of a sheet of paper:
a sample of EVA sheath (control);
a sample of EVA sheath and a sample of DINCH-plasticized PVC tubing;
a sample of EVA sheath and a sample of TOTM-plasticized PVC tubing.

As with the DINCH-plasticized PVC sheet, contact between a DINCH-plasticized PVC tubing and an EVA sheath has no influence on the UV transmittance of the EVA sheath.

Contact of TOTM-plasticized PVC tubing with an EVA sheath results in a drop of around 5% in the UV transmittance of the EVA sheath after 90 days. This drop rises to over 10% after one year.

### Example 4: Ability of Different Materials to Protect the UV Transmittance of the EVA Irradiation Bag

A single packaging pouch made of a transparent PET/PE composite film and a lacquered paper sheet is sealed so as to form two separate compartments. Each compartment contains a sample of EVA tubing (15 cm×15 cm) protected by an open sleeve. In addition, in the top compartment, two samples of DEHP-plasticized PVC (15 cm×15 cm) are sandwiched between the pouch and the EVA sheath protected by the sleeve.

Various materials were tested for manufacturing the protective sleeve: PET copolymer film, PET/PP copolymer film or spunbonded non-woven PET film.

TABLE 2

| | | UV-C transmittance (%) | | |
|---|---|---|---|---|
| | PVC- | Average (%) | Average (%) | Average (%) |
| | DEHP | T0 | T 1 month | 12 months |
| PET | Yes | 64.61 ± 0.66 | 64.68 ± 0.38 | 62.60 ± 1.16 |
| copolymer | | | | |
| PET | No | 64.61 ± 0.66 | 64.83 ± 0.63 | 63.44 ± 0.54 |
| copolymer | | | | |
| PET and PP | Yes | 64.61 ± 0.66 | 64.07 ± 0.58 | 63.76 ± 0.67 |
| copolymer | | | | |
| PET and PP | No | 64.61 ± 0.66 | 65.21 ± 0.41 | 64.05 ± 0.62 |
| copolymer | | | | |
| PET non- | Yes | 64.61 ± 0.66 | 62.86 ± 0.49 | 49.42 ± 1.53 |
| woven | | | | |
| PET non- | No | 64.61 ± 0.66 | 59.72 ± 0.66 | 55.64 ± 0.59 |
| woven | | | | |

These tests show that the PET copolymer film provides at least short-term protection for the irradiation bag against the diffusion of plasticizers (DEHP) and other contaminating substances from the packaging pouch.

The protective sleeve made of PET and PP copolymer ensures UV transmittance stability over time.

On the other hand, due to its high porosity, the PET spunbonded non-woven sleeve cannot maintain the UV transmittance of the EVA sheath.

1. Bag system (1) for the treatment of a biological fluid by electromagnetic irradiation, said bag system comprising at least one element consisting of an irradiation bag (2) intended to contain the biological fluid to be irradiated, said irradiation bag (2) being made of a material that is permeable to said electromagnetic radiation, said system comprising one or more other elements selected from the group consisting of at least one storage bag (8) and a tubing (9), at least one of said other elements being made of a polymer material formulated with at least one plasticizer, characterized in that it further comprises at least one protective film (26a, 26b) forming a barrier means to said plasticizer and covering said irradiation bag (2), said protective film (26a, 26b) being made of a gas-barrier polymer material having a permeability to oxygen of less than or equal to 100 cm³/m²/24 h at a temperature of 23° C. and a relative humidity of 50%.

2. Bag system according to claim 1, characterized in that the polymer material of the protective film (26a, 26b) is a water vapor barrier polymer material with a permeability to water vapor of less than or equal to 10 g/m²/24 h at a temperature of 23° C. and a relative humidity of 50%.

3. Bag system according to claim 1, characterized in that the protective film (26a, 26b) is transparent.

4. Bag system according to claim 1, characterized in that the protective film (26a, 26b) comprises at least one layer of polyester or polypropylene.

5. Bag system according to claim 1, characterized in that it comprises two protective films (26a, 26b) arranged on either side of the irradiation bag (2).

6. Bag system according to claim 5, characterized in that the two protective films (26a, 26b) together form a sleeve with at least one open end in which the irradiation bag (2) is arranged.

7. Bag system according to claim 1, characterized in that the electromagnetic irradiation treatment is a UV radiation treatment and the irradiation bag is made of a material permeable to UV radiation.

8. Bag system according to claim 1, characterized in that the irradiation bag (2) is free of photosensitive agent.

9. Bag system according to claim 1, characterized in that each of said other elements is made from a material or materials formulated without phthalate or terephthalate.

10. Bag system according to claim 9, characterized in that said other element(s) of the bag system comprise one or more elements selected from the group consisting of a storage bag (8), a sampling bag (11), tubing (6, 9, 12, 15), a connector (16), a clamp (18a, 18b, 18c, 18d), a vent (7a, 7b, 17a, 17b) and combinations thereof.

11. Bag system according to claim 9, characterized in that each of said other elements of said bag system is made of a material or materials other than polyvinyl chloride plasticized with di-2-ethylhexyl phthalate or di-2-ethylhexyl terephthalate.

12. Bag system according to claim 9, characterized in that each of said elements of the bag system is made either of polyvinyl chloride plasticized with a plasticizer selected from the group consisting of a cyclohexanedicarboxylic acid ester, a citrate ester, a trimellitate ester or a mixture thereof, or of a material other than plasticized polyvinyl chloride.

13. Assembly (20) for the treatment of a biological fluid by electromagnetic irradiation comprising a package (21) and a bag system (1) according to claim 1, characterized in that said bag system (1) is sterilely confined in said package (21).

14. Assembly according to claim 13, characterized in that the package (21) is formed of a porous non-woven sheet and of a transparent film sheet.

15. Use of a film (26a,26b) made of a gas-barrier polymer material having a permeability to oxygen of less than or equal to 100 cm³/m²/24 h at a temperature of 23° C. and a relative humidity of 50% as a barrier to releasable plasticizers of polyvinyl chloride.

16. Use of a film according to claim 15, characterized in that the film is made of a water vapor-barrier polymer material having a permeability to water vapor of less than or equal to 10 g/m²/24 h at a temperature of 23° C. and a relative humidity of 50%.
